(19) United States
(12) Patent Application Publication
(10) Pub. No.: US 2019/0275033 A1
(43) Pub. Date: Sep. 12, 2019

(54) USE OF ECM BIOMARKERS FOR DETERMINING THE TREATMENT ONSET WITH NINTEDANIB AND PIRFENIDONE

(71) Applicant: Boehringer Ingelheim International GmbH, Ingelheim am Rhein (DE)

(72) Inventors: Susanne STOWASSER, Loerzweiler (DE); Claudia DIEFENBACH, Stockach (DE)

(21) Appl. No.: 16/304,704

(22) PCT Filed: May 31, 2017
(86) PCT No.: PCT/EP2017/063178

§ 371 Date: Nov. 27, 2018

(30) Foreign Application Priority Data
Jun. 1, 2016 (EP) 16172487.7
Sep. 2, 2016 (EP) 16187089.4

(51) Int. Cl. A61K 31/496; A61P 11/00; A61K 31/4418; G01N 33/573

(52) U.S. Cl. CPC A61K 31/496; G01N 33/573; A61K 31/4418; A61P 11/00

(57) ABSTRACT

Disclosed is a method for treatment of idiopatic pulmonary fibrosis and progressive fibrosing interstitial lung disease (PF-ILD). In one embodiment, the method of the invention comprises administering nintedanib, or pharmaceutical acceptable salt thereof, and pirfenidone, or a pharmaceutical acceptable salt thereof to a patient in need thereof.

### BACKGROUND TO THE INVENTION

IPF

Idiopathic pulmonary fibrosis (IPF) belongs to a large group of more than 200 lung diseases known as interstitial lung diseases (ILDs), which are characterized by the involvement of the lung interstitium, the tissue between the air sacs of the lung.

Idiopathic pulmonary fibrosis (IPF) is a rare disease of unknown aetiology that is characterized by progressive fibrosis of the interstitium of the lung, leading to decreasing lung volume and progressive pulmonary insufficiency. The course of the disease in individual patients is variable: some patients progress rapidly, others have periods of relative stability punctuated by acute exacerbations and others progress relatively slowly. Acute exacerbations of IPF are events of respiratory deterioration of unidentified cause that occur in 5-10% of patients annually and are associated with a very poor outcome. IPF is most prevalent in middle aged and elderly patients, and usually presents between the ages of 40 and 70 years. The median life expectancy in IPF patients after diagnosis is 2 to 3 years. The latest update on clinical practice guideline for the treatment of IPF, jointly issued in 2015 by the American Thoracic Society (ATS), European Respiratory Society (ERS), Japanese Respiratory Society (JRS) and Latin American Thoracic Association (ALAT) has provided a conditional recommendation for treatment with nintedanib or pirfenidone for the majority of IPF patients, taking into account individual patient values and preferences. Conventional IPF treatments such as n-acetylcysteine (NAC), corticosteroids, cyclophosphamide, cyclosporine and azathioprine are not approved treatments for IPF, and their efficacy is questionable or even harmful. Nonpharmacological therapies such as pulmonary rehabilitation and long-term oxygen therapy are recommended for some patients, but their efficacy in patients with IPF has not been established. Lung transplant has been shown to positively impact survival in patients with IPF. Although the number of patients transplanted due to IPF has increased steadily over the last years, the scarce availability of donor organs, as well as the comorbidities and advanced age preclude many patients from referral to lung transplant. Pirfenidone, a compound which demonstrated anti-fibrotic activity in non-clinical models, was first licensed in Japan in 2008 based on two local trials which showed a reduced decline of vital capacity under treatment with the compound. In the international Phase III CAPACITY program, pirfenidone demonstrated efficacy on the primary FVC lung function endpoint in only one of two confirmatory trials. The additional confirmatory ASCEND Phase III trial requested by FDA met the primary endpoint of change from baseline FVC % predicted. Pirfenidone is also licensed since February 2011 for the treatment of mild to moderate IPF in the European Union and since October 2014 for the treatment of IPF in the United States of America. It is also licensed in several other countries. Nintedanib is a small molecule intracellular tyrosine kinase inhibitor which has demonstrated anti-fibrotic and anti-inflammatory activity in preclinical models. The two replicate Phase III INPULSIS trials and the Phase II TOMORROW trial consistently showed positive results for the efficacy of nintedanib 150 mg twice daily versus placebo in patients with IPF. Both INPULSIS trials showed that nintedanib reduced the annual rate of decline in FVC (mL/year) by approximately 50%, consistent with slowing disease progression. Based on these three clinical trials, nintedanib was approved for the treatment of IPF in the USA in October 2014, in the European Union in January 2015 and in Japan in July 2015. As of 15 Apr. 2017, nintedanib has been authorised in the indication of treatment of IPF in sixty countries (including Canada, Switzerland, Russia, Australia, Chile, Ecuador and Taiwan). It has been submitted for marketing authorization in other countries across the world.

Nintedanib

Nintedanib, the compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone is an innovative compound having valuable pharmacological properties, especially for the treatment of oncological diseases, immunologic diseases or pathological conditions involving an immunologic component, or fibrotic diseases.

The chemical structure of this compound is depicted below as Formula A.

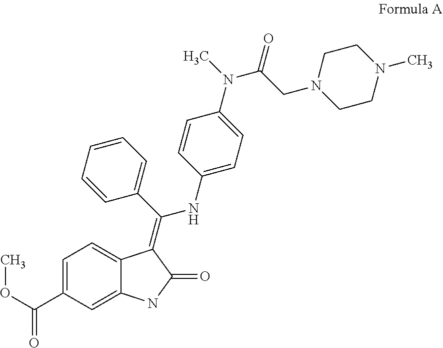 [SMILES: COC(=O)c1ccc2c(c1)NC(=O)/C2=C(\Nc1ccc(N(C)C(=O)CN2CCN(C)CC2)cc1)c1ccccc1]

The base form of this compound is described in WO 01/27081, the monoethanesulpho-nate salt form is described in WO 2004/013099 and various further salt forms are presented in WO 2007/141283. The use of this molecule for the treatment of immuno-logic diseases or pathological conditions involving an immunologic component is being described in WO 2004/017948, the use for the treatment of oncological diseases is being described in WO 2004/096224 and the use for the treatment of fibrotic diseases is being described in WO 2006/067165.

The monoethanesulphonate salt form of this compound presents properties which makes this salt form especially suitable for development as medicament. The chemical structure of 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate is depicted below as Formula A1.

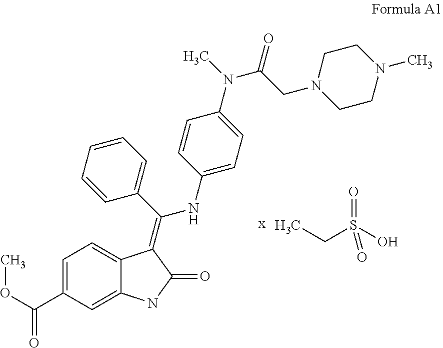

Preclinical studies have shown that this compound is a highly potent, orally bioavailable inhibitor of vascular endothelial growth factor receptors (VEGFRs), platelet-derived growth factor receptors (PDGFRs) and fibroblast growth factor receptors (FGFRs) and the antifibrotic potential ofVEGFR, PDGFR, and FGFR inhibition with nintedanib has been evaluated in a series of preclinical studies. Data showing the kinase specificity profile of nintedanib has been published by Hilberg et al., Cancer Res. 2008; 68: 4774-82, i.e. regarding angiogenesis and fibrosis-related kinases such as VEGFR, PDGFR and FGFR, regarding Src family kinases related to inflammation and proliferation such as Src, Lck and Lyn, as well as other kinases, e.g. FLT-3, IGF1R, InsR, EGFR, HER2, CDK1, CDK2 and CDK4. Nintedanib was shown to inhibit PDGFR-α and -β activation and proliferation of normal human lung fibroblasts in vitro and to inhibit PDGF-BB-, FGF-2-, and VEGF-induced proliferation of human lung fibroblasts from patients with IPF and control donors. Nintedanib attenuated PDGF- or FGF-2-stimulated migration of lung fibroblasts from patients with IPF and inhibited transforming growth factor (TGF)-β-induced fibroblast to myofibroblast transformation of primary human lung fibroblasts from IPF patients (Hostettler et al., Respir. Res. 2014; 15: 157; Wollin et al., J. Pharmacol. Exp. Ther. 2014; 349:209-20). The polypharmacology of nintedanib has been described by L. Wollin et al., Eur. Respir. J. 2015; 45: 1434-1445. In two different mouse models of IPF, nintedanib exerted anti-inflammatory effects as shown by significant reductions in lymphocyte and neutrophil counts in the bronchoalveolar lavage fluid, reductions in inflammatory cytokines and reduced inflammation and granuloma formation in histological analysis of lung tissue. IPF mouse models also revealed nintedanib-associated antifibrotic effects as shown by significant reductions in total lung collagen and by reduced fibrosis identified in histological analyses.

Posology: Nintedanib the recommended dose is 150 mg nintedanib twice daily administered approximately 12 hours apart. The amount of nintedanib to be administered is calculated on the free base while it is actually formulated as monoethanesulphonate. The 100 mg twice daily dose is only recommended to be used in patients who do not tolerate the 150 mg twice daily dose.

If a dose is missed, administration should resume at the next scheduled time at the recommended dose.

If a dose is missed the patient should not take an additional dose. The recommended maximum daily dose of 300 mg should not be exceeded.

Dose adjustments: In addition to symptomatic treatment if applicable, the management of adverse reactions to nintedanib (see Ofev® EPAR of the EMA, sections 4.4 and 4.8) could include dose reduction and temporary interruption until the specific adverse reaction has resolved to levels that allow continuation of therapy. Nintedanib treatment may be resumed at the full dose (150 mg twice daily) or a reduced dose (100 mg twice daily). If a patient does not tolerate 100 mg twice daily, treatment with nintendanib should be discontinued. In case of interruptions due to aspartate aminotransferase (AST) or alanine aminotransferase (ALT) elevations >3× upper limit of normal (ULN), once transaminases have returned to baseline values, treatment with Ofev may be reintroduced at a reduced dose (100 mg twice daily) which subsequently may be increased to the full dose (150 mg twice daily) (see EPAR sections 4.4 and 4.8).

Hepatic Impairment:

Nintedanib is predominantly eliminated via biliary/faecal excretion (>90%). Exposure increased in patients with hepatic impairment (Child Pugh A, Child Pugh B; see EPAR section 5.2). In patients with mild hepatic impairment (Child Pugh A), the recommended dose of Ofev is 100 mg twice daily approximately 12 hours apart. In patients with mild hepatic impairment (Child Pugh A), treatment interruption or discontinuation for management of adverse reactions should be considered. The safety and efficacy of nintedanib have not been investigated in patients with hepatic impairment classified as Child Pugh B and C. Treatment of patients with moderate (Child Pugh B) and severe (Child Pugh C) hepatic impairment with Ofev is not recommended (see EPAR section 5.2).

Pirfenidone:

Pirfenidone is 5-methyl-1-phenyl-2(1H)-Pyridinone having the CAS number 53179-13-8. The chemical structure of this compound is depicted below as Formula B:

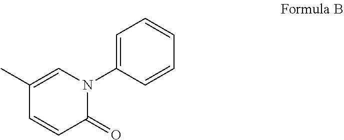

Pirfenidone is marketed as Esbriet® in capsules of 267 mg pirfenidone.

Esbriet is used to treat adults with mild to moderate idiopathic pulmonary fibrosis (IPF) in the EU.

Treatment Regimen for Adults:

Upon initiating treatment, the dose should be titrated to the recommended daily dose of nine capsules per day over a 14-day period as follows:
Days 1 to 7: one capsule, three times a day (801 mg/day)
Days 8 to 14: two capsules, three times a day (1602 mg/day)
Day 15 onward: three capsules, three times a day (2403 mg/day)

The recommended daily dose of Esbriet for patients with IPF is three 267 mg capsules three times a day with food for a total of 2403 mg/day. Doses above 2403 mg/day are not recommended for any patient.

Patients who miss 14 consecutive days or more of Esbriet treatment should re-initiate therapy by undergoing the initial 2-week titration regimen up to the recommended daily dose. For treatment interruption of less than 14 consecutive days, the dose can be resumed at the previous recommended daily dose without titration.

Although nintedanib and pirfenidone can be considered a standard of care for patients diagnosed with IPF, it remains unclear when to start and when to stop treatment with either of the drugs, given the unpredictability of clinical course in the individual patient. With the introduction of nintedanib in the treatment algorithm of IPF, there is an additional need to further characterize its profile in patients at an early disease stage, i.e. in patients with limited lung volume impairment, and to address the question when to start treatment in these patients. Currently, many physicians apply a wait and watch strategy for these patients as there are no markers to predict the individual course in a given patient or response to treatment which may result in a delay of treatment initiation. Identifying biomarkers to predict the clinical course and benefits of therapy for a given patient early in the course of the disease remains one of the most urgent and relevant challenges in patient management. Moreover, the diagnosis of IPF itself may be a time consuming procedure. Basically it requires the confirmation of the histological or imaging pattern of usual interstitial pneumonia (UIP) absence of alternative causes such as drug toxicity, environmental exposure (e.g. asbestos) or collagen vascular disease (e.g. scleroderma, rheumatoid arthritis). There is a need for a diagnostic concept that early indicates patients who will benefit from treatment with nintedanib.

### SUMMARY OF THE INVENTION

One embodiment of the invention is s compound selected from the group consisting of nintedanib and pharmaceutical acceptable salts thereof, and pirfenidone and pharmaceutical acceptable salts thereof, for use in the treatment of idiopatic pulmonary fibrosis, wherein the onset of the treatment is determined by the determination CRPM content of a body sample of the patient at least at two consecutive time points and wherein the treatment starts if the rate of the change of concentration of CRPM is greater than 1.7 ng/ml, more preferred greater than 1 ng/ml per month, most preferred greater than 0 ng/ml per month.

The invention allows an early identification of those IPF patients that particularly benefit from the treatment because their disease will further progress.

A further embodiment of the invention is a compound selected from the group consisting of nintedanib and pharmaceutical acceptable salts thereof for use in the treatment of progressive fibrosing interstitial lung disease (PF-ILD), wherein the onset of the treatment is determined by the determination CRPM content of a body sample of the patient at least at two consecutive time points and wherein the treatment starts if the rate of the change of concentration of CRPM is greater than 1.7 ng/ml, more preferred greater than 1 ng/ml per month, most preferred greater than 0 ng/ml per month.

The invention early indicates patients who will benefit from treatment with nintedanib.

In a further embodiment of the invention PF-ILD is idiopathic non-specific interstitial pneumonia (iNSIP).

In a further embodiment of the invention PF-ILD is unclassifiable idiopathic interstitial pneumonia (unclassifiable IIP).

In a further embodiment of the invention PF-ILD is idiopathic pneumonia with autoimmune features (IPAF).

In a further embodiment of the invention PF-ILD is chronic hypersensitivity pneumonitis (CHP).

In a further embodiment of the invention PF-ILD is environmental/occupational fibrosing lung diseases.

In a further embodiment of the invention PF-ILD is systemic sclerosis interstitial lung disease (SSc-ILD).

In a further embodiment of the invention PF-ILD is or rheumatoid arthritis interstitial lung disease (RA-ILD).

### DETAILED DESCRIPTION OF THE INVENTION

CRPM Determination

CRPM means C-reactive protein degraded by matrix metalloprotease 1/8 (MMP-1/8) that has been evaluated in the PROFILE study. In this study serum samples were prospectively collected at baseline, 1 month, 3 months, and 6 months and were analysed for a panel of novel matrix metalloprotease (MMP)-degraded ECM proteins, by ELISA-based, neoepitope assay. 11 neoepitopes were tested in a discovery cohort of 55 patients to identify biomarkers of sufficient rigour for more detailed analyses. Eight were then further assessed in a validation cohort of 134 patients with 50 age-matched and sex-matched controls. Changes in biomarker concentrations were related to subsequent progression of idiopathic pulmonary fibrosis (defined as death or decline in forced vital capacity >10% at 12 months after study enrolment) using a repeated measures model. The PROFILE study is registered on ClinicalTrials.gov, numbers NCT01134822 and NCT01110694, see JENKINS et al., Lancet Respir Med (2015), http://dx.doi.org/10.1016/S2213-2600(15)00048-X, page 1-11. This study revealed that for CRPM a rate greater than 0 ng/ml per month conferred a HR of 2.16 (95% CI 1.15-4.07), whereas a rate greater than 1 ng/ml per month resulted in an HR 4.08 (2.14-7.8), and a rate greater than 1.7 ng/ml/month was associated with an HR 6.61 (95% CI 2.74-15.94). Hazard ratio represents the mortality risk in participants with rising neoepitope concentrations relative to those with stable or falling concentrations (see, page 7, col. 2, 3^{rd} paragraph of JENKINS et al.)

The C-reactive protein (CRP) is considered the prototypical acute phase reactant in human and is produced in response to a variety of clinical conditions including infection, inflammation and tissue injury. During acute phase stimulus the serum concentration of CRP approaches a 1000 to 10.000-fold increase within 24-48 hours and decreases just as rapidly to the low normal concentration of a few μg/mL. CRP is upregulated in both situations of acute and chronic inflammatory diseases, however it is a non-specific biochemical marker due to its upregulation in all inflammatory diseases the prototypical acute phase reactant in human and is produced in response to a variety of clinical conditions including infection, inflammation and tissue injury (VOLANAKIS, Mol Immunol 2001; 38: 189-97-DU CLOS, Ann Med 2000; 32: 274-8—HIRSCHFIELD, PEPYS, QJM 2003; 96:793-807).

The determination of CRPM in serum samples follows the procedure disclosed in SKJøT-ARKIL et al., Clinical and Experimental Rheumatology 2012; 30: 371-379, in particular 373-375:
“In vitro cleavage of CRP
Purified CRP from human serum (Alpha Diagnostics) was cleaved with MMP-1, MMP-9, cathepsin K, cathepsin S (Calbiochem, VWR), MMP-3, MMP-8 (Abcam), A Disintegrin And Metalloproteinase with a Thrombospondin motif (ADAMTS)-1, and -8 (Abnova). The proteases were activated according to the manufacturers's instructions. Each cleavage was performed separately by mixing 200 μg CRP and 2 μg of activated enzymes in MMP buffer (100 mM Tris-HCl, 100 mM NaCl, 10 mM CaCl₂, 2 mM ZnOAc, pH 8.0), cathepsin buffer (50 mM NaOAc, 20 mM L-cystine, pH=5.5) or aggrecanase buffer (50 mM tris-HCl, 10 mM NaCl, 10 mM CaCl2, pH=7.5). As control 200 μg CRP was mixed with MMP buffer only. Each aliquot was incubated for three days at 37° C. All MMP cleavages were terminated using GM6001 (Sigma-Aldrich) and all cathepsin and aggrecanase cleavages using E64 (Sigma-Aldrich). Finally the cleavage was verified by visualization using the SilverXpress® Silver Staining Kit (Invitrogen) according to the manufacturers' instructions.

Peptide identification by MS
The cleavage products were purified and desalted using reversed phase (RP) micro-columns (Applied Biosystems) prior to nanoLC-MS-MS analysis as describes in literature (see THINGHOLM & LARSEN: Methods Mol Biol 2009; 527: 57-66, xi.28). The purified peptides were resuspended in 100% formic acid, diluted with H₂O and loaded directly onto a 18 cm RP capillary column using a nano-Easy-LC system (Proxeon, Thermo Scientific). The peptides were eluted using a gradient from 100% phase A (0.1% formic acid) to 35% phase B (0.1% formic acid, 95% acetonitrile) over 43 min directly into an LTQ-Orbitrap XL mass spectrometer (Thermo Scientific).
For each MS scan (Orbitrap, resolution of 60000, 300-1800 Da range) the five most abundant precursor ions were selected for fragmentation (CID). The raw data files were converted to mgf files and searched in Mascot 2.2 using Proteome Discoverer (Thermo Scientific).
Peptides with a mascot probability score p<0.05 were further analysed.
Selection of peptide for immunisations
The first six amino acids of each free end of the sequences identified by MS were regarded as a neoepitope generated by the protease in question. All protease-generated sequences were analysed for homology and distance to other cleavage sites and then blasted for homology using the NPS@: network protein sequence analysis (COMBET, BLANCHET, GEOURJON, DELEAGE, Trends Biochem Sci 2000; 25: 147-50).

Immunisation procedure
Six 4-6 week old Balb/C mice were immunised subcutaneously in the abdomen with 200 μL emulsified antigen (50 μg per immunisation) using Freund's incomplete adjuvant (KAFVFPKESD-GGC-KLH and GNFEGSQSLV-GGC-OVA (Chinese Peptide Company, Beijing, China)). Immunisations were continued until stable titer levels were obtained. The mouse with the highest titer was selected for fusion and boosted intravenously with 50 μg immunogen in 100 μL 0.9% sodium chloride solution three days before isolation of the spleen for cell fusion. The fusion procedure has been previously described (GEFTER, MARGULIES, SCHARFF, Somatic Cell Genet 1977; 3: 231-6).
Characterization of clones
The potential sequences KAFVFP and GNFEGS, named CRP-MMP and CRPCAT respectively, were selected for antibody generation. Native reactivity and peptide binding of the monoclonal antibodies were evaluated by displacement of human serum in a preliminary indirect ELISA using biotinylated peptides (KAFVFPKESD-K-Biotin or GNFEGSQSLV-K-Biotin) on a streptavidin coated microtitre plate and the supernatant from the growing monoclonal hybridoma. Tested were the specificities of clones to the free peptide (KAFVFPKESD or GNFEGSQSLV), a non-sense peptide, and the elongated peptide (RKAFVFPKESD or GGNFEGSQSLV). Isotyping of the monoclonal antibodies was performed using the Clonotyping System-HRP kit (Southern Biotech). The selected clones were purified using Protein G columns according to manufacturer's instructions (GE Healthcare Life Science).
Assay protocol
The selected monoclonal antibodies were labelled with horseradish peroxidase (HRP) using the Lightning link HRP labelling kit according to the instructions of the manufacturer (Innovabioscience). A 96-well streptavidin plate was coated with 1.25 ng/mL KAFVFPKESD-K-Biotin (CRP-MMP assay) or 0.40 ng/mL GNFEGSQSLVK-Biotin (CRP-CAT assay) dissolved in assay buffer (25 mM Tris, 1% BSA, 0.1% Tween-20, pH 7.4) and incubated 30 minutes at 20° C. 20 μL of free peptide calibrator or sample were added in duplicates to appropriate wells, followed by 100 μL of conjugated monoclonal antibody (1A7-HRP or 3H8-HRP) and incubated 1 hour at 20° C. Finally, 100 μL tetramethylbenzinidine (TMB) (Kem-En-Tec) was added and the plate was incubated 15 minutes at 20° C. in the dark. All the above incubation steps included shaking at 300 rpm. After each incubation step the plate was washed five times in washing buffer (20 mM Tris, 50 mM NaCl, pH 7.2). The TMB reaction was stopped by adding 100 μL of stopping solution (1% HCl) and measured at 450 nm with 650 nm as the reference. A master calibrator, prepared from the synthetic free peptide accurately quantified by amino acid analysis, was used as a calibration curve and plotted using a 4-parametric mathematical fit model.
Technical evaluation and specificity
From 2-fold dilutions of quality control (QC) serum samples, linearity was calculated as a percentage of recovery of the 100% sample. The lower limit of detection was determined from 21 zero samples (i.e. buffer) and calculated as the mean+3× standard deviation. The inter- and intra-assay variation was determined by 12 independent runs of 8 QC samples, with each run consisting of two replicas of double determinations.
The stability of serum samples was measured for three samples, which have been frozen and thawed for one to ten times. The developed CRP-MMP and CRPCAT ELISAs were evaluated using the materials described under “In vitro cleavage”, where CRP was cleaved by different MMPs, cathepsins and aggrecanases. The materials were diluted 1:10 in the ELISA.
CRP-MMP, CRP-CAT vs. total CRP in patients
CRP-MMP, CRP-CAT and full-length human CRP (Quantikine, R&D System) were assessed in serum from patients diagnosed with AS and compared to healthy sex- and age-matched controls from the Department of Medicine 3 of the University of Erlangen-Nuremberg.
Serum samples were retrieved from patients diagnosed with ankylosing spondylitis (AS) according to the modified New York criteria and from sex- and age-matched non-diseased controls. BASDAI and mSASSS was registered for the each of the AS patients.
The samples were diluted 1:4 in the CRP-MMP assay and in the CRP-CAT assay. The study was approved by the Ethics Committee of the University of Erlangen-Nuremberg and conformed to the principles outlined in the Declaration of Helsinki. Written informed consent was obtained from each person.
Statistics
Serum levels of the individual biomarkers between AS patients and non-diseased controls were compared using two-sided non-parametric Wilcoxon rank sum test. Correlations between the biomarkers were investigated by non-parametric Spearman's test. Area under the curve was measured with use of Receiver Operating Characteristic (ROC). The biomarkers were investigated in odds ratios (extrapolated from weighted levels: lowest value in the population was set at 0 and highest at 1) where all subject were classified as having normal (within SD of the mean of the normal population) or high (>SD) levels of the biomarker. Results were considered statistically significant if p<0.05.”

The upper and lower limits of the quantification of CRPM (MMP degraded CRP-1/8) are 3.2 and 110.0 ng/ml, respectively and the intra/inter assay variability is <11.1% and <20.8% (JENKINS et al., Supplementary Table and Figure Legends).

PF-ILD Definition

The scientific working hypothesis is that the response to lung injury in fibrosing ILDs includes the development of fibrosis which becomes progressive, self-sustaining and independent of the original clinical association or trigger.

Based on the similarity in both, their biologic and clinical behaviors i.e. self-sustaining fibrosis and progressive decline in lung function and early mortality, it is considered justified to group patients with PF-ILD together regardless of the original ILD diagnosis.

Non-Clinical Development

Nintedanib was explored in pre-clinical model systems of lung fibrosis and in more specific models of SSc-ILD and rheumatoid arthritis-associated ILD (RA-ILD).

Although the initiation of the fibrotic lung pathology in these model systems is different, progressive fibrotic lung pathology with proliferation, migration and transformation of fibroblasts to the pathogenic myofibroblast is the final common pathway. These similarities and the mode of action of nintedanib directed against the proliferation, migration and transformation of fibroblasts strongly support the rationale for the use of nintedanib in the treatment of patients with PF-ILD. The efficacy of nintedanib in animal models of lung fibrosis, SSc-ILD and RA-ILD at comparable doses suggests comparable dosing in patients with PF-ILD. A comprehensive non-clinical development program was completed during the development of nintedanib in the indication IPF; for details, see New Drug Application (NDA) 205832 for OFEV® (nintedanib) capsules.

Pharmacodynamics

During the pre-clinical pharmacodynamic exploration of nintedanib in in vitro and in vivo models of IPF the mode of action in lung fibrosis was characterized. Nintedanib inhibited the proliferation and migration of human lung fibroblasts from patients with IPF. Data are shown in Huang et al., Ann. Rheum. Dis. 2016; 75: 883-90, also disclosing attenuation of bleomycin-induced skin fibrosis in mice by therapeutic nintedanib treatment. Furthermore, it has been shown that Nintedanib normalises skin and lung fibrosis as well as lung vascular remodelling in Fra2+/−Mice, a genetic model of SSc-ILD. Nintedanib has the potential to interfere at multiple steps in the pathobiology of SSc, as may be recognized from Denton et al., Nat. Clin. Pract. Rheumatol. 2006; 1:134-44.

Nintedanib also attenuated the transformation of lung fibroblasts to myofibroblasts. Fibroblast proliferation, migration and transformation are fundamental processes in the common final path of several diseases resulting in lung fibrosis such as IPF but also SSc-ILD and RA-ILD. In three animal models of lung fibrosis nintedanib demonstrated anti-fibrotic and anti-inflammatory activity regardless if administered in a preventive or therapeutic dosing regimen. In these studies nintedanib reduced the histology score of inflammation, granuloma formation and fibrosis in lung sections, attenuated the accumulation of lymphocytes in the bronchoalveolar lavage fluid (BALF), reduced interleukin (IL)-10, the chemokine CXCL1/KC, tissue inhibitor of metalloproteinases (TIMP)-1 and the collagen content in lung tissue and blocked messenger ribonucleic acid (mRNA) expression of fibrosis-related marker genes such as transforming growth factor (TGF)-β1 and procollagen 1.

In experiments with dermal fibroblasts from patients with SSc, nintedanib inhibited migration and proliferation, reduced the expression of extracellular matrix markers and attenuated transformation to myofibroblast. In four animal models of SSc and SSc-ILD with different features nintedanib effectively attenuated skin and lung fibrosis, reduced extracellular matrix deposition in skin and lung, attenuated myofibroblast accumulation in skin and lung and reduced dermal thickening. Nintedanib also reduced dermal microvascular endothelial cell apoptosis. Additionally, nintedanib effectively attenuated pulmonary vascular remodelling in an animal model of SSc by reducing the number of vascular smooth muscle cells and occluded pulmonary vessels.

In an animal model of RA-ILD nintedanib was found to have pharmacodynamic effects in transgenic SKG mice stimulated with zymosan to induce an arthritis pathology and lung fibrosis (Redente et al., Am. J. Respir. Crit. Care Med. 2016; 193: A4170: Nintedanib Reduces Pulmonary Fibrosis In A Model Of Rheumatoid Arthritis Associated Interstitial Lung Disease; see Example 1 together with FIGS. 1 to 4). In this model the arthritis score increased up to 6 weeks after zymosan administration. The score remained high up to week 10 and then slowly decreased. However, lung fibrosis which was determined by the lung collagen (hydroxyproline as a marker of collagen deposition) content was increased at week 10 after zymosan administration and further increased till week 16. Early treatment with nintedanib during week 5-11 after zymosan administration attenuated arthritis pathology and improved lung function (static lung compliance) but had no effect on lung collagen deposition. Late nintedanib treatment during week 10-16 only resulted in a reduction of the arthritis score if the score was normalized to the last measurement before the start of the nintedanib treatment. However, the late treatment reduced the lung fibrosis demonstrated by a reduction of collagen deposition in the lung. In SKG mice treated with zymosan nintedanib seem to trigger a slight but selective inflammation in the lung. Whether this inflammation is helpful to resolve the fibrosis or detrimental remains open.

Nintedanib was also tested in a mouse model of collagen-induced arthritis (CIA). Nintedanib treatment was started 13 days after the induction of the arthritis pathology with type II collagen mixed with Freund's adjuvant. The treatment with nintedanib was continued for 5 weeks. The arthritis score further increased during the 5 weeks of nintedanib treatment. Nintedanib had no attenuating effect on the arthritis score.

An overview of the pre-clinical exploration of nintedanib in different in vivo models of lung fibrosis is presented in Table 1.

TABLE 1
Overview of the pre-clinical exploration of nintedanib in animal model of lung fibrosis

| Model | Bleomycin-induced | | Silica-induced lung | | Bleomycin-induced | | Skin and | Rheumatoid arthritis | |
|---|---|---|---|---|---|---|---|---|---|
| system | lung fibrosis in mice | | fibrosis in mice | | lung fibrosis in rats | | lung fibrosis | and lung fibrosis in | |
| | | | | | | | in transgenic | SKG mice | |
| | | | | | | | Fra-2 mice | | |
| Model | Lung epithelial cell | | Lung epithelial cell | | Lung epithelial cell | | Resembles | Resembles aspects of | |
| character- | injury-induced lung | | injury-induced ongoing | | injury-induced lung | | aspects of | arthritic joint | |
| istics | inflammation and | | lung inflammation and | | inflammation and | | skin and | inflammation and | |
| | fibrosis | | progressive fibrosis | | fibrosis | | progressive | progressive lung | |
| | | | | | | | lung fibrosis | fibrosis | |
| | | | | | | | including | | |
| | | | | | | | microvascular | | |
| | | | | | | | disease and | | |
| | | | | | | | pulmonary | | |
| | | | | | | | hypertension | | |
| | | | | | | | with typical | | |
| | | | | | | | lesions | | |
| Treatment | preventive | therapeutic | preventive | therapeutic | preventive | therapeutic | therapeutic | early | late |
| Effects of | Lung | Lung | Lung | Lung | Lung | Lung | Skin and | Arthritic | Arthritic |
| nintedanib | fibrosis↓ | fibrosis↓ | fibrosis↓ | fibrosis↓ | fibrosis | fibrosis | lung | score↓ | score↓* |
| | Lung | Lung | Lung | Lung | (Ashcroft | (Ashcroft | myofibroblast | Static | Lung |
| | inflam- | inflam- | inflam- | inflam- | score)↓ | score)↓ | count↓ | lung | hydroxiproline↓ |
| | mation↓ | mation↓ | mation↓ | mation↓ | | | Dermal | compli- | BAL |
| | IL-1β↓ | Lung | Lung | Lung | | | thickness↓ | ance↑ | lymphocytes and |
| | TIMP-1↓ | collgen↓ | collagen↓ | collagen↓ | | | Hydroxiproline↓ | Lung | neutrophils↑ |
| | BALF | IL-1β↓ | IL-1β↓ | IL-1β↓ | | | ECM↓ | tissue | Lung tissue |
| | lympho- | TIMP-1↓ | KC↓ | IL-6↓ | | | Vessel wall | B220 + | inflammatory |
| | cytes↓ | BALF | TIMP-1↓ | KC↓ | | | thickness↓ | B-cells↑ | macrophages↑ |
| | | lympho- | BALF | TIMP-1↓ | | | Occluded | CD103 + | |
| | | cytes↓ | neutrophils + | BALF | | | vessels↓ | Dendritic | |
| | | | lympho- | neutrophils + | | | VSMC↓ | cells↑ | |
| | | | cytes↓ | lympho- | | | MVEC | Monocytes↓ | |
| | | | | cytes↓ | | | apoptosis↑ | Neutrophils↑ | |

↓significant reduction (independent of dose used);
↑significant increase (independent of dose used);
*If the data is normalised to the last scoring before the nintedanib treatment starts.
BALF, bronchoalveolar lavage fluid; IL-1β; interleukin-1β; IL-6, interleukin-6; TIMP-1, tissue inhibitor of matrix metalloproteinase 1; KC, chemokine CXCL1/KC; ECM, extracellular matrix; VSMC, vascular smooth muscle cells; MVEC, microvascular endothelial cells.

To summarize, it has been shown that
nintedanib at clinically relevant concentrations inhibits human lung fibroblast migration, proliferation, contraction and potentially fibroblast to myofibroblast transformation,
nintedanib reduces markers of extracellular matrix and myofibroblast activation and blocks proliferation and migration of dermal fibroblasts from patients with SSc,
nintedanib demonstrates anti-fibrotic, anti-angiogenic and anti-inflammatory activity in various animal model of lung fibrosis induced by different triggers,
the translation into the clinics reveals good efficacy of nintedanib in patients with IPF.

Preclinical exploration and clinical translation in further interstitial lung diseases is ongoing. SENSCIS™ (Safety and Efficacy of Nintedanib in Systemic SCleroslS) study investigating nintedanib in people with systemic sclerosis who also develop interstitial lung disease started in December 2015.

The main fibrosing ILDs in which progressive behaviour is present include:
IPF,
iNSIP,
unclassifiable IIP,
autoimmune ILD that includes connective tissue disease-associated ILD (CTD-ILD) [mainly rheumatoid arthritis ILD (RA-ILD) and systemic sclerosis ILD (sSSc-ILD)] and idiopathic pneumonia with autoimmune features (IPAF),
chronic hypersensitivity pneumonitis (CHP) and
environmental/occupational fibrosing lung diseases

With the exception of nintedanib and pirfenidone which are available for patients with IPF, there is no approved therapy for PF-ILD. Apart from IPF and SSc-ILD, no prospective, controlled clinical trials have been performed in other ILDs.

Definition of the Patient Population for the Assessment of Nintedanib in PF-ILD

PF-ILD is defined of the set of patients with features of diffuse fibrosing lung disease of >10% extent on HRCT and whose lung function and respiratory symptoms or chest imaging have worsened despite treatment with unapproved medications used in clinical practice to treat ILD e.g. corticosteroid, azathioprine (AZA), mycophenolate mofetil (MMF), N-acetyl cysteine (NAC), rituximab, cyclophosphamide, cyclosporine, tacrolimus.

The onset of the treatment is determining the CRPM content of a body sample of the patient at least two times and wherein the rate of the change of concentration of CRPM is greater than 1.7 ng/ml per month, preferred greater than 1 ng/ml per month, most preferred greater than 0 ng/ml per month.

### Example 1: Nintedanib Reduces Pulmonary Fibrosis in a Model of Rheumatoid Arthritis Associated Interstitial Lung Disease

(Redente et al., Am. J. Respir. Crit. Care Med. 2016; 193: A4170):

INTRODUCTION: Rheumatoid arthritis preferentially affects women and approximately 40.70% of patients have lung abnormalities and involvement. From this group approximately 20% will develop rheumatoid arthritis-associated interstitial lung disease (RA-ILD). Female SKG mice are arthritis-prone and authentically reproduce human RA-ILD:
Penetrance of both the joint disease (100%) and lung disease (20%)
The development of lung histopathologic and leukocyte infiltration patterns similar to cellular nonspecific interstitial pneumonia in humans,
The development of pulmonary fibrosis in a subset of affected mice
The development of circulating auto-antibodies against citrulinated epitopes.

RATIONALE: SKG mice, genetically prone to develop autoimmune arthritis, also develop a pulmonary interstitial pneumonia that resembles human cellular and fibrotic non-specific interstitial pneumonia. Experiments were carried out to test whether the early treatment of arthritic SKG animals with nintedanib would prevent the development of interstitial pneumonia and whether late intervention of SKG animals with RA and interstitial pneumonia would result in a reduction of their fibrotic burden.

METHODS: The effect of nintedanib in female SKG mice (50 mice/group) receiving 5 mg of zymosan to induce arthritis and associated interstitial pneumonia was investigated. Beginning at week 5 or 11 after zymosan, mice received a daily gavage of 60 mg/kg nintedanib or saline as a control. Animals were harvested after 6 weeks of nintedanib treatment and fibrotic lung disease was assessed by measuring hydroxyproline levels, lung physiology measurements including static compliance and Masson's trichrome staining. Inflammation in the lungs was measured by BAL (bronchoalveolar lavage) cellularity and in enzymatically-digested lungs. Arthritis of joints and digits was scored weekly.

RESULTS: Therapeutic delivery of nintedanib for six weeks to mice with established arthritis showed a significant reduction of lung collagen measured by hydroxyproline and staining for collagen. Mice receiving nintedanib beginning at week 5 also had a significant reduction in their development of arthritis. Treatment with nintedanib induced a small but significant increase in CD4+ T-cells and B220+ B-cells. Mice receiving nintedanib beginning at week 5 but not at week 11, had a significant increase in lung neutrophils and dendritic cells, but there were no changes in macrophage numbers.

CONCLUSIONS: The results indicate that the therapeutic delivery of nintedanib significantly reduces the pulmonary fibrosis in arthritic SKG mice. In addition, early intervention with nintedanib significantly reduced the development of arthritis in SKG mice. This study impacts the rheumatoid arthritis and interstitial lung diseases field by helping to identify a therapeutic treatment that may improve both diseases. The presented model replicates the incidence and characteristics of human RA-ILD.

Results are shown in FIGS. 1 to 4.

### SHORT DESCRIPTION OF THE FIGURES

FIG. 1: Arthritis Inflammation Score, effect of Nintedanib on established joint swelling. Joint swelling and damage was not altered by daily dosing with nintedanib beginning 10 weeks after zymosan injection:

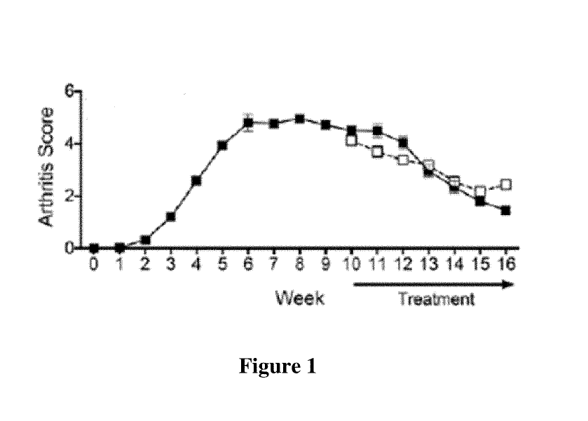

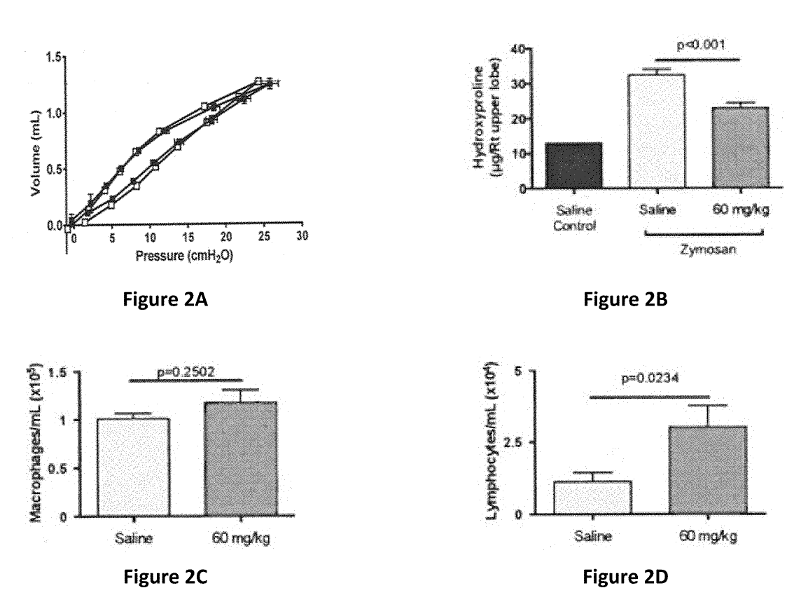

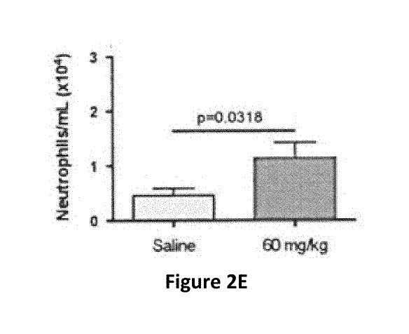

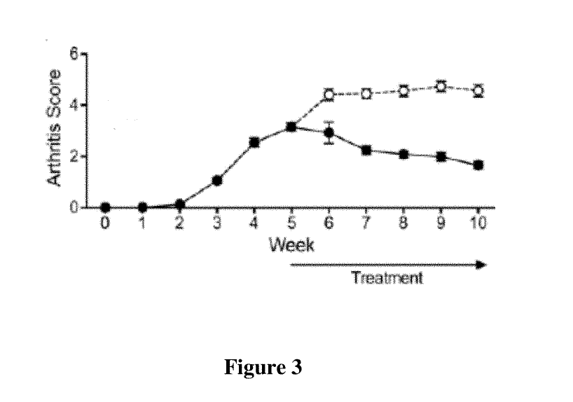

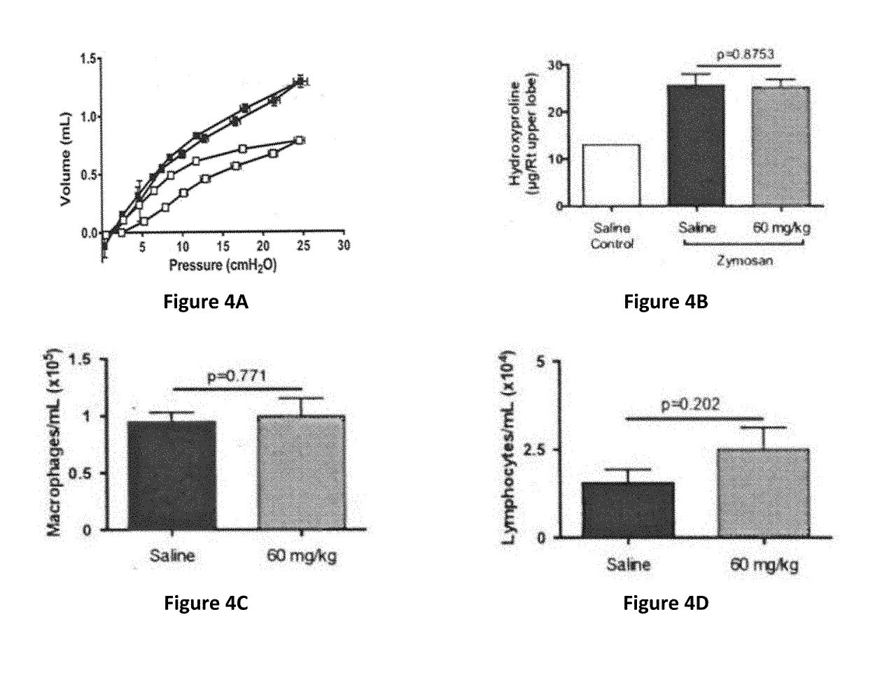

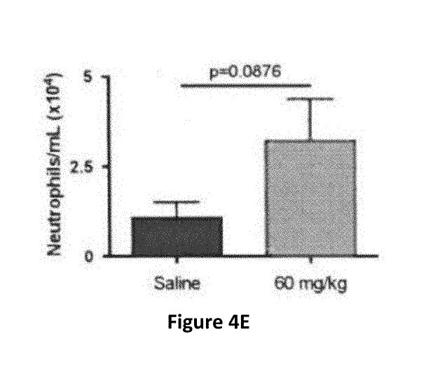

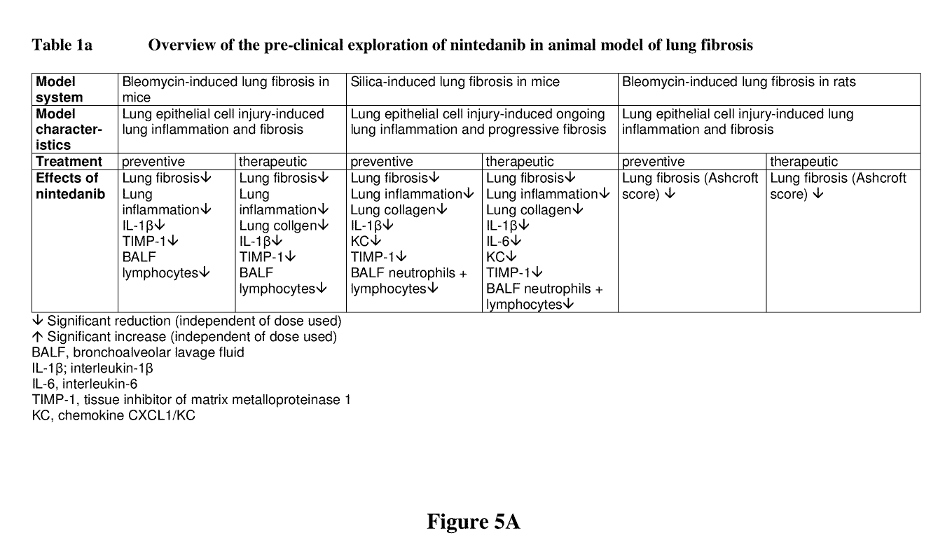

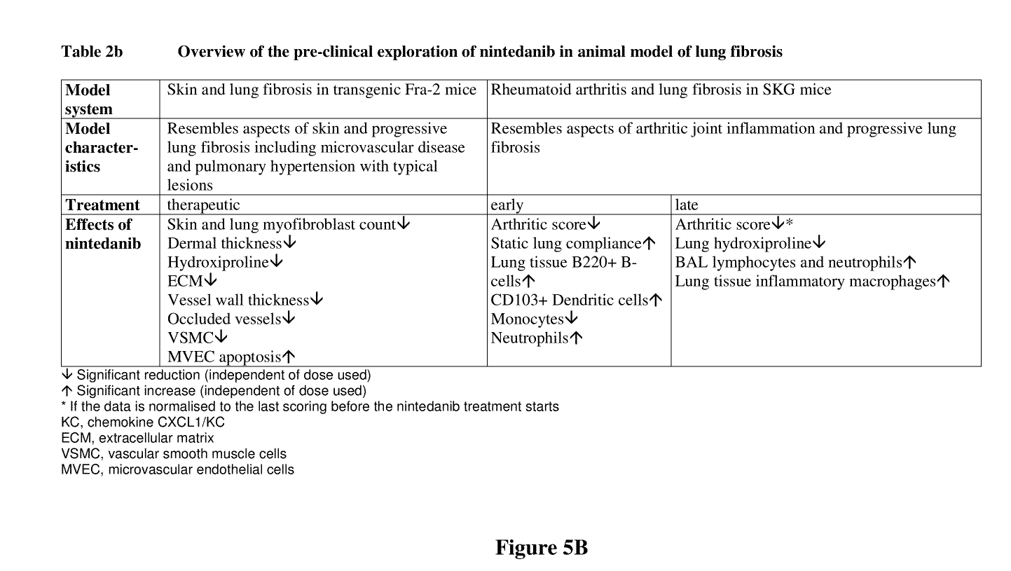

To determine the effect of nintedanib on established joint and pulmonary disease, mice received a daily oral gavage of nintedanib or saline beginning 10 weeks post-zymosan injection. There was no significant change in weight or percent survival with nintedanib compared with saline at week 16. Joint swelling, assessed by visual arthritis score, was first detected 2-3 weeks post-zymosan injection and gradually increased before peaking in severity 6-10 weeks post-zymosan injection. Thereafter, the severity of joint swelling declined but remained elevated in mice 16 weeks post-zymosan injection. Administration of nintedanib, starting at week 10, slightly increased the resolution of joint swelling compared with saline-treated animals as shown in FIG. 1.

FIG. 2: Nintedanib did not alter lung compliance in lungs with established lung disease: A modest but statistically significant decline in static lung compliance was observed in zymosan-injected SKG mice in the saline group but there was no significant difference in static lung compliance in nintedanib-treated animals as analyzed by pressure volume curves (A). There was no significant difference in the total number of BAL cells numbers (P=0.2223, data not shown) or in alveolar macrophages (P=0.2502) in nintedanib-treated versus saline-treated animals (D). There was a significant decrease in collagen levels measured by hydroxyproline (C) after treatment of nintedanib. A small but significant elevation in the number of lymphocytes (E) and neutrophils (F) was detected in the nintedanib-treated cohort (P=0.0234 and P=0.0318, respectively) in the SKG model of lung disease.

FIG. 3: Arthritis Inflammation Score, effect of Nintedanib on developing joint swelling. Joint swelling and damage was reduced by daily dosing with nintedanib beginning 5 weeks after zymosan injection:

Treatment with nintedanib led to a reversal of joint swelling when compared with saline-treated mice that became most apparent after the mice had been receiving nintedanib for 1 week. Thereafter, the arthritis scores in nintedanib-treated mice began to decline progressively over the ensuing 4 weeks. In contrast, the arthritis score progressively increased in mice receiving saline.

FIG. 4: Effect of Nintedanib on developing interstitial pneumonia:

In contrast to mice with established disease, an upward shift was observed in the pressure-volume curves of nintedanib-treated animals, indicating an increase in static lung compliance (A). Hydroxyproline levels (a measure of collagen) were not significantly altered in the cohort of mice receiving nintedanib compared with saline (C) during the development of lung disease. There was no significant difference in the total number of alveolar macrophages in nintedanib-treated versus saline-treated animals (D). There was a trend towards increased lymphocytes (E) and neutrophils (F) in the BAL after nintedanib.

### Example 2: Effect of Nintedanib on Biomarkers of ECM Turnover in Patients with IPF and Limited FVC Impairment

A 12-week, double blind, randomised, placebo controlled, parallel group trial followed by a single active arm phase of 40 weeks evaluating the effect of oral nintedanib 150 mg twice daily on change in biomarkers of extracellular matrix (ECM) turnover in patients with idiopathic pulmonary fibrosis (IPF) and limited forced vital capacity (FVC) impairment to investigate the effect of nintedanib on various extracellular matrix (ECM) turnover biomarkers and the predictive value of change in those ECM biomarkers on disease progression.

Main Inclusion criteria: Male or female patients aged ≥40 years at Visit 1 (screening); IPF diagnosis based upon ATS/ERS/JRS/ALAT 2011 guideline within 3 years of Visit 0; HRCT performed within 18 months of Visit 0; confirmation of diagnosis by central review of chest HRCT and surgical lung biopsy (later if available) prior to randomisation; FVC ≥80% predicted of normal at Visit 1 (screening).

Posology: 300 mg daily (150 mg bid) with possibility to reduce total daily dose to 200 mg (100 mg bid) to manage adverse events (AEs).

Primary Endpoint: Rate of change (slope) in blood CRPM from baseline to week 12.

Key Secondary Endpoint: Proportion of patients with disease progression as defined by absolute FVC (% predicted) decline ≥10% or death until week 52.

Secondary Endpoints: Rate of change (slope) in blood C1M from baseline to week 12;

Rate of change (slope) in blood C3M from baseline to week 12.

Further Endpoints (selected): Rate of change (slope) in blood CRPM, C1M and C3M from week 12 to week 52.

Safety criteria: Adverse events (especially SAE and other significant AE), physical examination, weight measurements, 12 lead electrocardiogram, vital signs and laboratory evaluations.

Statistical methods: Random coefficient regression models for continuous endpoints, Log rank tests, Kaplan-Meier plots and Cox regressions for time to event endpoints, logistic regression models or other appropriate methods for binary endpoints.

1. A method of treating idiopathic pulmonary fibrosis, the method comprising administering to a patient in need thereof a compound selected from the group consisting of nintedanib and a pharmaceutical acceptable salt thereof, and pirfenidone, and a pharmaceutical acceptable salt thereof, wherein the onset of the treatment is determined by the determination of C-reactive protein degraded by matrix metalloprotease 1/8 (CRPM) content of a body sample, of the patient at least at two consecutive time points and wherein the treatment starts if the rate of the change of concentration of CRPM is greater than 0 ng/ml per month.

2. The method of claim 1, wherein the rate of the change of concentration of CRPM is greater than 1 ng/ml per month.

3. The method of claim 1, wherein the rate is greater than 1.7 ng/ml per month.

4. The method of claim 1, wherein nintedanib is in the form of its monoethanesulphonate salt.

5. A method for treating progressive fibrosing interstitial lung disease (PF-ILD) in a patient in need thereof, comprising administering nintedanib or a pharmaceutical acceptable salt thereof, wherein the onset of the treatment is determined by the determination CRPM content of a body sample of the patient at least at two consecutive time points and wherein the treatment starts if the rate of the change of concentration of CRPM is greater than 0 7 ng/ml per month.

6. The method of claim 5, wherein the rate is greater than 1 ng/ml per month.

7. The method of claim 5, wherein the rate is greater than 1.7 ng/ml per month.

8. The method of claim 5, wherein nintedanib is in the form of its monoethanesulphonate salt.

9. The method of claim 5, wherein the PF-ILD is idiopathic non-specific interstitial pneumonia (iNSIP).

10. The method of claim 5, wherein the PF-ILD is unclassifiable idiopathic interstitial pneumonia (unclassifiable IIP).

11. The method of claim 5, wherein the PF-ILD is idiopathic pneumonia with autoimmune features (IPAF).

12. The method of claim 5, wherein the PF-ILD is chronic hypersensitivity pneumonitis (CHP).

13. The method of claim 5, wherein the PF-ILD is environmental/occupational fibrosing lung diseases.

14. The method of claim 5, wherein the PF-ILD is SSc-ILD.

15. The method of claim 5, wherein the PF-ILD is RA-ILD.

16. The method of claim 1, wherein the body sample is serum.

17. The method of claim 1, wherein the body sample is plasma.

18. The method of claim 1, wherein the rate is determined on the basis of a time interval of 4 to 12 weeks.

19. The method of claim 1, wherein the rate is determined on the basis of a time interval of about 12 weeks.

20. The method of claim 1, wherein the rate is determined on the basis of a time interval of 12 weeks.
